(19) United States
(12) Patent Application Publication
(10) Pub. No.: US 2024/0382768 A1
(43) Pub. Date: Nov. 21, 2024

(54) COMPUTER-ASSISTED PROGRAMMING OF NEUROMODULATION THERAPY

(71) Applicant: Boston Scientific Neuromodulation Corporation, Valencia, CA (US)

(72) Inventors: Adarsh Jayakumar, Valencia, CA (US); Eric Manning, Santa Monica, CA (US)

(21) Appl. No.: 18/652,645

(22) Filed: May 1, 2024

Related U.S. Application Data
(60) Provisional application No. 63/466,522, filed on May 15, 2023.

(51) Int. Cl. A61N 1/372; A61N 1/05; A61N 1/36; G16H 40/63

(52) U.S. Cl. CPC A61N 1/37276; A61N 1/0551; A61N 1/36062; A61N 1/36071; A61N 1/36135; A61N 1/36157; A61N 1/3616; A61N 1/36171; A61N 1/36175; A61N 1/36185; A61N 1/37229; A61N 1/37241; A61N 1/37247; G16H 40/63

(57) ABSTRACT

Systems and methods for programming a neuromodulation therapy to a neuromodulation device are disclosed. An exemplary system comprises an electrostimulator and a programmer device operable by a user to program the electrostimulator. The programmer device includes a user interface and a controller circuit. The controller circuit collects user commands and contextual information, including a sequence of programming instructions received from the user during a programming session. The controller circuit determines a programming quality indicator using the user commands and contextual information, and generates an individualized programming workflow to guide the user to test and program a therapy setting. The electrostimulator can deliver a neuromodulation therapy in accordance with the user programmed therapy setting.

### CLAIM OF PRIORITY

This application claims the benefit of U.S. Provisional Application No. 63/466,522 filed on May 15, 2023, which is hereby incorporated by reference in its entirety.

### TECHNICAL FIELD

This document relates generally to medical devices, and more particularly, to computer-assisted programming of a medical device to provide neuromodulation therapy.

### BACKGROUND

Neuromodulation (or “neural neuromodulation”, also referred to as “neurostimulation” or “neural stimulation”) has been proposed as a therapy for a number of conditions. Often, neuromodulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS systems have been used as a therapeutic modality for the treatment of chronic pain syndromes. PNS has been used to treat chronic pain syndrome and incontinence, with a number of other applications under investigation. FES systems have been applied to restore some functionality to paralyzed extremities in spinal cord injury patients. DBS can be used to treat a variety of diseases or disorders.

Stimulation systems, such as implantable electrostimulators, have been developed to provide therapy for a variety of treatments. An implantable electrostimulator can include a pulse generator and one or more leads each including a plurality of stimulation electrodes. The stimulation electrodes are in contact with or near target tissue to be stimulated, such as nerves, muscles, or other tissue. The control module generates a control signal to the pulse generator, which generates electrostimulation pulses that are delivered by the electrodes to the target tissue in accordance with an electrode configuration and a set of stimulation parameters.

Neuromodulation therapy can be programmed into an electrostimulator by a user (e.g., a clinician or an authorized device user) using a programmer device. One example of such neuromodulation therapy is paresthesia-based spinal cord stimulation (SCS) to treat chronic pain. Paresthesia is a sensation such as tingling, prickling, heat, cold, etc. that accompanies the SCS therapy. Alternatively, SCS may be delivered with reduced stimulation intensity below a paresthesia threshold to avoid inducing paresthesia, yet still achieve analgesia effect and clinically effective pain relief. Such paresthesia-free SCS, also known as sub-perception SCS, generally uses stimulation pulses at higher frequencies to achieve the paresthesia-free effect, which may consume more power than paresthesia-based SCS. Proper programming of therapies such as paresthesia-based or paresthesia-free SCS is important to achieve therapeutic benefits while avoiding or minimizing side effects of such neuromodulation therapies.

### SUMMARY

Programming an electrostimulator using a programmer device generally requires a user to select or provide values for a number of therapy parameters such as amplitude, frequency, pulse width, waveforms, electrodes (cathode and anode) selected for delivering electrostimulation energy. The programming may also involve selecting, or providing values for, a sensing parameter used for sensing a physiological signal from the patient, such as a neural activity signal or an electrophysiological signal. Sensing a physiological signal while providing an electrostimulation therapy provides a feedback mechanism that may help assess patient responses to the therapy, which may help regulate or optimize a closed-loop therapy. However, finding an optimal or desired closed-loop therapy for the patient and programming such therapy to the electrostimulator generally require testing of multiple device parameters (e.g., sensing parameter and therapy parameters) and various combinations thereof, which can be time consuming and add significant complexity. Moreover, as more advanced neuromodulation therapies are developed and made available to the patient, the level of sophistication involved in neuromodulation therapy testing, optimization, and programming continues to rise. Some users (e.g., clinicians or authorized device users) may not be familiar with, thus do not use or know how to best use, certain device features pertaining to advanced neural activity signal sensing and processing, feature extraction and selection, and neuromodulation therapy programming. Even with proper training, some users may not readily envision how a different programmed value of a device feature would affect the therapeutic outcome in a patient. As a result, some advanced device features may be significantly underused if not misused. Some users may not consistently customize their programming workflow to search for individualized optimal therapies. The process of searching for and programming an optimal therapy for the patient can be inefficient. Furthermore, the longer the user stays within a given programming session, the more battery power is consumed, especially when testing and optimization of certain programmable parameters requires active communication between the electrostimulator system (e.g., battery-powered implantable device) and the programmer device.

The present inventors have recognized an unmet need for computer-assisted, individualized programming of neuromodulation therapy. According to one embodiment, an electrostimulation system comprises an electrostimulator configured to provide a neuromodulation therapy to a patient, and a programmer device operable by a user to program the electrostimulator. The programmer device includes a user interface and a controller circuit. The controller circuit collects user commands and contextual information during a programming session, including a sequence of programming instructions received from the user via the user interface. The controller circuit can determine a programming quality indicator using the user commands and contextual information. Based on the programming quality indicator, the controller circuit generates an individualized programming workflow that can guide the user to test and program a therapy setting. The electrostimulator can provide the neuromodulation therapy in accordance with the user programmed therapy setting.

Various examples discussed in this document may improve the efficiency and quality of testing and programming neuromodulation therapy to a patient. In an embodiment, a programming quality indicator can be generated using user commands and contextual information during a programming session. The programming quality indicator indicates a level of efficiency of user's programming as well as how the patient responds to neuromodulation therapy. An individualized programming workflow generated based on such programming quality indicator may expedite the search process for an individualized optimal neuromodulation therapy for the patient, reduce therapy programming time for users of different levels of experience with the device features, improve their work efficiency, and reduce overall operation cost. The reduced programming time may also reduce battery power consumption and extend battery life for battery-powered neuromodulation devices. Additionally, in accordance with some examples as described herein, the individualized programming workflow can be automatically generated or updated, and provide more direct and effective guidance to the device users to help them become familiar with advanced and complex devices features. This reduces the burden and cost of training the clinicians and device users, while at the same time promotes more consistent and efficient use of such device features.

Example 1 is a system, comprising: an electrostimulator configured to provide a neuromodulation therapy to a patient; and a programmer device operable by a user to program the electrostimulator, the programmer device including: a user interface; and a controller circuit configured to: collect user commands and contextual information during a programming session, including a sequence of programming instructions from the user via the user interface; determine a programming quality indicator using the collected user commands and contextual information; and based at least in part on the programming quality indicator, generate an individualized programming workflow to guide the user to test and program a therapy setting to the electrostimulator; wherein the electrostimulator is configured to provide the neuromodulation therapy in accordance with the user programmed therapy setting.

In Example 2, the subject matter of Example 1 optionally includes the sequence of programming instructions that can include one or more of a timing of a programmable parameter being selected, a frequency of a programmable parameter being selected over a specific time period, or an order of selecting multiple programmable parameters, wherein the individualized programming workflow includes selection or deselection of a programmable parameter, or a specific order of selecting multiple programmable parameters.

In Example 3, the subject matter of any one or more of Examples 1-2 optionally includes the controller circuit that can be configured to receive image or video data from an imaging system during the programming session, and to extract information about the sequence of programming instructions from the received image or video data.

In Example 4, the subject matter of any one or more of Examples 1-3 optionally includes the user commands and contextual information collected during the programming session that further includes time elapsed during an entirety or a portion of the programming session, including time spent when the programmer device is in active communication with the electrostimulator during the programming session.

In Example 5, the subject matter of any one or more of Examples 1˜4 optionally include the user commands and contextual information collected during the programming session that further includes an estimate of power consumption during an entirety or a specific portion of the programming session, including an estimate of power consumption when the programmer device is in active communication with the electrostimulator during the programming session.

In Example 6, the subject matter of Example 5 optionally includes the individualized programming workflow that can include an ordered selection of multiple programmable parameters based on their respective dependencies on an active communication during the programming session.

In Example 7, the subject matter of Example 6 optionally includes the controller circuit that can be configured to identify a first group of programmable parameters that require active communication during the programming session, and a second group of programmable parameters that do not require active communication during the programming session, and to generate the individualized programming workflow including prioritizing selection of second group of programmable parameters over the first group of programmable parameters.

In Example 8, the subject matter of any one or more of Examples 1-7 optionally includes the user commands and contextual information collected during the programming session that further includes patient responses indicative of a therapeutic effect or a side effect of the neuromodulation therapy.

In Example 9, the subject matter of Example 8 optionally includes at least one physiological sensor configured to sense the patient responses to the neuromodulation therapy.

In Example 10, the subject matter of any one or more of Examples 1-9 optionally includes the controller circuit that can be configured to determine the programming quality indicator based on a comparison of the sequence of programming instructions to a reference sequence of programming instructions.

In Example 11, the subject matter of any one or more of Examples 1-10 optionally includes the programming quality indicator that can include a programming quality score computed using a weighted combination of various sources of the user commands and contextual information collected during the programming session.

In Example 12, the subject matter of any one or more of Examples 1-11 optionally includes the controller circuit that can be configured to determine the programming quality indicator by applying a trained machine learning model to the user commands and contextual information collected during the programming session.

In Example 13, the subject matter of any one or more of Examples 1-12 optionally includes the controller circuit that can be configured to present on the user interface the individualized programming workflow, including to prompt the user to select or deselect a programmable parameter, or to select multiple programmable parameters in a specific order.

In Example 14, the subject matter of Example 13 optionally includes the programmable parameter that can include at least one of a signal acquisition parameter, a signal analysis parameter, or a neuromodulation therapy parameter.

In Example 15, the subject matter of any one or more of Examples 1-14 optionally includes the programmer device that can include a communication circuit to communicate with a cloud computing system, and to use one or more cloud services provided by the cloud computing system to perform one or more operations including collecting the user commands and contextual information during the programming session, determining the programming quality indicator, or generating the individualized programming workflow.

Example 16 is a method of programming an electrostimulator using a programmer device to provide electrostimulation to a patient, the method comprising: receiving user commands and contextual information during a programming session, including a sequence of programming instructions from a user via a user interface; determining a programming quality indicator using the user commands and contextual information; based at least in part on the programming quality indicator, generating an individualized programming workflow to guide the user to test and program a therapy setting to the electrostimulator; and delivering a neuromodulation therapy to the patient via the electrostimulator in accordance with the user programmed therapy setting.

In Example 17, the subject matter of Example 16 optionally includes the sequence of programming instructions from the user during the programming session that can include one or more of a timing of a programmable parameter being selected, a frequency of a programmable parameter being selected over a specific time period, or an order of selecting multiple programmable parameters, wherein the individualized programming workflow includes selection or deselection of a programmable parameter, or a specific order of selecting multiple programmable parameters.

In Example 18, the subject matter of any one or more of Examples 16-17 optionally includes user commands and contextual information during the programming session that further includes at least one of: time elapsed during an entirety or a portion of the programming session, including time spent when the programmer device is in active communication with the electrostimulator during the programming session; or an estimate of power consumption during an entirety or a specific portion of the programming session, including an estimate of power consumption when the programmer device is in active communication with the electrostimulator during the programming session.

In Example 19, the subject matter of Example 18 optionally includes the individualized programming workflow that can include an ordered selection of multiple programmable parameters based on their respective dependencies on an active communication during the programming session, such that a first group of programmable parameters that do not require active communication during the programming session are selected prior to a second group of programmable parameters that require active communication during the programming session.

In Example 20, the subject matter of any one or more of Examples 16-19 optionally includes the programming quality indicator that can include a programming quality score computed using a weighted combination of various sources of the received user commands and contextual information during the programming session.

In Example 21, the subject matter of any one or more of Examples 16-20 optionally include presenting on the user interface the individualized programming workflow, including prompting the user to select or deselect a programmable parameter, or selecting multiple programmable parameters in a specific order, wherein the programmable parameter includes at least one of a signal acquisition parameter, a signal analysis parameter, or a neuromodulation therapy parameter.

In Example 22, the subject matter of any one or more of Examples 16-21 optionally includes: establishing a communication between the programmer device and a cloud-computing system and receiving cloud-based services from the cloud-computing system; and using the received cloud-based services to perform one or more operations including collecting the user commands and contextual information during the programming session, determining the programming quality indicator, or generating the individualized programming workflow.

The description that follows will generally focus on the use of the invention within a Spinal Cord Stimulation (SCS) systems. However, the present invention may find applicability with any implantable neurostimulator device system, including DBS system, Vagus Nerve Stimulation (VNS) system, Sacral Nerve Stimulation (SNS) systems, and the like. For example, apparatus and methods for detecting (and maintaining) exceptionally small evoked neural activities as described herein can be used to detect evoked neural activities in closed-loop DBS therapy, or therapees of other regions of the nervous system. The following examples illustrate various aspects of the examples described herein.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates, by way of example and not limitation, an Implantable Pulse Generator (IPG) useable for Spinal Cord Stimulation (SCS).

FIG. 2 illustrates, by way of example and not limitation, an example of stimulation pulses producible by an IPG.

FIG. 3 illustrates, by way of example and not limitation, use of an External Trial Stimulator (ETS) useable to provide stimulation before implantation of an IPG.

FIG. 4 illustrates, by way of example and not limitation, various external devices capable of communicating with and programming stimulation in an IPG and ETS.

FIG. 5 illustrates, by way of example and not limitation, a Graphical User Interface (GUI) for setting or adjusting stimulation parameters.

FIGS. 6A-6B illustrate, by way of example and not limitation, schematics of electrode configurations and stimulation waveforms that may be used in fast-acting sub-perception neuromodulation therapy.

FIG. 7 illustrates, by way of example and not limitation, a neuromodulation system for providing or adjusting a neuromodulation therapy in accordance with parameters programmed through a computer-assisted individualized programming workflow.

FIG. 8 illustrates an example of user commands and contextual information collected in a programming session and used for generating an individualized programming workflow.

FIGS. 9A-9B illustrate, by way of example and not limitation, at least a portion of a graphical user interface that displays an individualized programming workflow to guide the user to test and program a device parameter to an electrostimulator.

FIG. 10 illustrates an example of a cloud-based neuromodulation system for analyzing multiple programming sessions and optimizing workflows for programming respective electrostimulators across multiple patients.

FIG. 11 is a flow chart illustrating, by way of example and not limitation, a method for programming an electrostimulator to provide electrostimulation to a patient.

FIG. 12 illustrates generally a block diagram of an example machine upon which any one or more of the techniques (e.g., methodologies) discussed herein may perform.

### DETAILED DESCRIPTION

This document describes systems and methods for programming neuromodulation therapy to a neuromodulation device. An exemplary system comprises an electrostimulator and a programmer device operable by a user to program the electrostimulator. The programmer device includes a user interface and a controller circuit. The controller circuit collects user commands and contextual information, including a sequence of programming instructions received from the user during a programming session. The controller circuit determines a programming quality indicator using the user commands and contextual information, and generates an individualized programming workflow to guide the user to test and program a therapy setting. The electrostimulator can deliver a neuromodulation therapy in accordance with the user programmed therapy setting.

The following detailed description of the present subject matter refers to the accompanying drawings which show, by way of illustration, specific aspects and examples in which the present subject matter may be practiced. These examples are described in sufficient detail to enable those skilled in the art to practice the present subject matter. Other examples may be utilized and structural, logical, and electrical changes may be made without departing from the scope of the present subject matter. References to “an”, “one”, or “various” examples in this disclosure are not necessarily to the same example, and such references contemplate more than one example. The following detailed description is, therefore, not to be taken in a limiting sense, and the scope is defined only by the appended claims, along with the full scope of legal equivalents to which such claims are entitled.

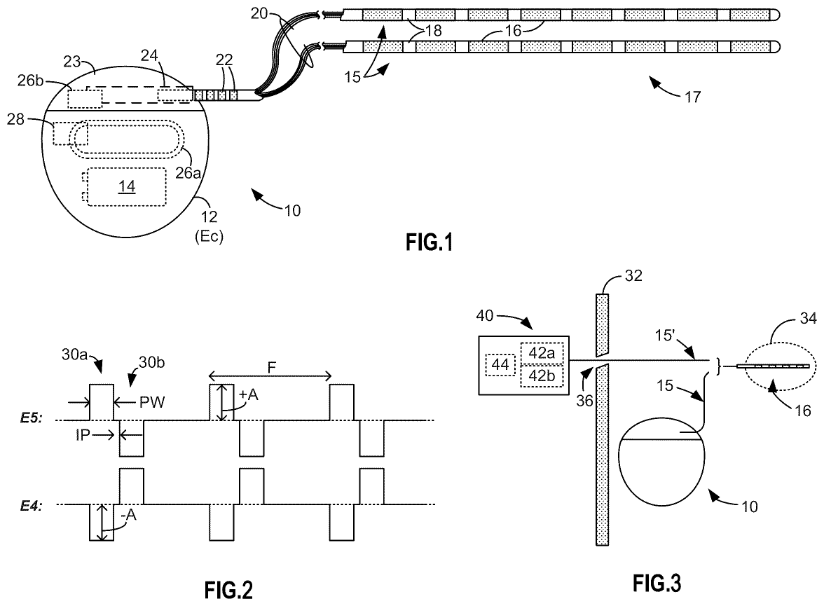

FIG. 1 illustrates, by way of example and not limitation, an Implantable Pulse Generator (IPG) 10 useable for Spinal Cord Stimulation (SCS). The IPG 10 includes a biocompatible device case 12 that holds the circuitry and battery 14 necessary for the IPG to function. The IPG 10 is coupled to electrodes 16 via one or more electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren't shown.

By way of example and not limitation, in the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient's spinal column on the right and left sides of the spinal cord midline. The proximal contacts 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The IPG leads 15 can be integrated with and permanently connected the case 12 in other IPG solutions. The goal of SCS therapy is to provide electrical stimulation from the electrodes 16 to alleviate a patient's symptoms, most notably chronic back pain.

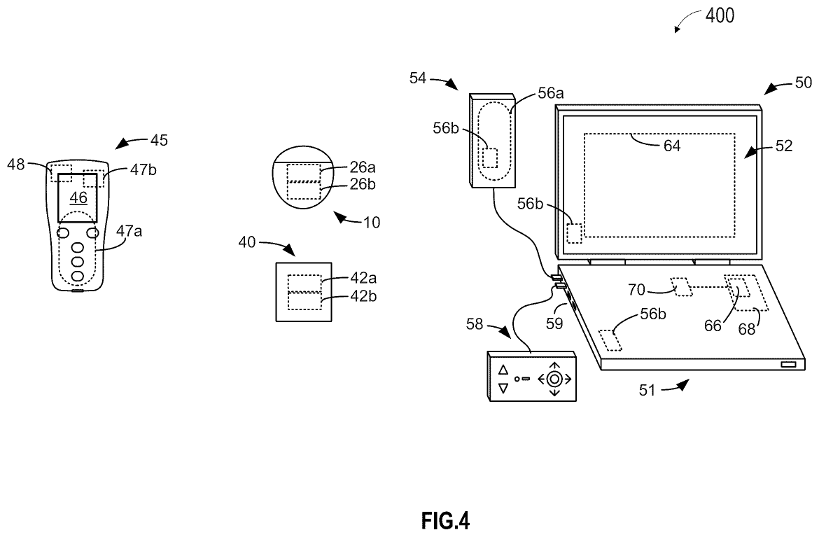

The IPG 10 can include an antenna 26a allowing it to communicate bi-directionally with a number of external devices, as shown in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radiofrequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in the IPG 10 is typically provided by pulses, as shown in FIG. 2. Stimulation parameters typically include the amplitude of the pulses (A; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

FIG. 2 illustrates, by way of example and not limitation, an example of stimulation pulses producible by an IPG, such as the IPG 10. In this example, the electrode E5 has been selected as an anode, and thus provides pulses which source a positive current of amplitude +A to the tissue. Electrode E4 has been selected as a cathode, and thus provides pulses which sink a corresponding negative current of amplitude-A from the tissue. This is an example of bipolar stimulation, in which only two lead-based electrodes are used to provide stimulation to the tissue (one anode, one cathode). However, more than one electrode may act as an anode at a given time, and more than one electrode may act as a cathode at a given time (e.g., tripolar stimulation, quadripolar stimulation, etc.).

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially connected DC-blocking capacitor, see, e.g., U.S. Patent Application Publication 2016/0144183, which will charge during the first phase 30a and discharged (be recovered) during the second phase 30b. In the example shown, the first and second phases 30a and 30b have the same duration and amplitude (although opposite polarities), which ensures the same amount of charge during both phases. In some examples, the second phase 30b may be charged balance with the first phase 30a if the integral of the amplitude and durations of the two phases are equal in magnitude. The width of each pulse, PW, is defined here as the duration of first pulse phase 30a, although pulse width could also refer to the total duration of the first and second pulse phases 30a and 30b as well. Note that an interphase period during which no stimulation is provided may be provided between the two phases 30a and 30b.

The IPG 10 includes stimulation circuitry 28 that can be programmed to produce the stimulation pulses at the electrodes as defined by the stimulation program. Stimulation circuitry 28 can for example comprise the circuitry described in U.S. Patent Application Publications 2018/0071513 and 2018/0071520, or in U.S. Pat. Nos. 8,606,362 and 8,620,436. The entirety of such references are incorporated herein by reference.

FIG. 3 illustrates, by way of example and not limitation, use of an External Trial Stimulator (ETS) 40 useable to provide stimulation, and at least a portion of external trial stimulation environment that may precede implantation of an IPG 10 in a patient. During external trial stimulation, stimulation can be tried on a prospective implant patient without going so far as to implant the IPG 10. Instead, one or more trial leads 15′ are implanted in the patient's tissue 32 at a target location 34, such as within the spinal column as explained earlier. The proximal ends of the trial lead(s) 15′ exit an incision 36 and are connected to an External Trial Stimulator (ETS) 40. The ETS 40 generally mimics operation of the IPG 10, and thus can provide stimulation pulses to the patient's tissue as explained above. See, e.g., U.S. Pat. No. 9,259,574, disclosing a design for an ETS. The ETS 40 is generally worn externally by the patient for a short while (e.g., two weeks), which allows the patient and his clinician to experiment with different stimulation parameters to try and find a stimulation program that alleviates the patient's symptoms (e.g., pain). If external trial stimulation proves successful, trial lead(s) 15′ are explanted, and a full IPG 10 and lead(s) 15 are implanted as described above; if unsuccessful, the trial lead(s) 15′ are simply explanted.

Like the IPG 10, the ETS 40 can include one or more antennas to enable bi-directional communications with external devices, explained further with respect to FIG. 4. Such antennas can include a near-field magnetic-induction coil antenna 42a, and/or a far-field RF antenna 42b, as described earlier. ETS 40 may also include stimulation circuitry 44 able to form the stimulation pulses in accordance with a stimulation program, which circuitry may be similar to or comprise the same stimulation circuitry 28 present in the IPG 10. ETS 40 may also include a battery (not shown) for operational power.

FIG. 4 illustrates at least a portion of a neuromodulation system 400. The neuromodulation system 400 comprises the IPG 10 and the ETS 40 as described above with reference to FIGS. 1-3, and various external devices capable of communicating with and programming stimulation in the IPG 10 and the ETS 40, including a patient, hand-held external remote controller (RC) 45, and a clinician programmer (CP) 50. Both the RC 45 and the CP 50 can be used to send a stimulation program to the IPG 10 or ETS 40—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 40 is currently executing. Devices 45 and 50 may also receive information from the IPG 10 or ETS 40, such as various status information, etc.

The RC 45 can be as described in U.S. Patent Application Publication 2015/0080982 for example, and may comprise a dedicated controller configured to work with the IPG 10. The RC 45 may also comprise a general purpose mobile electronics device such as a mobile phone which has been programmed with a Medical Device Application (MDA) allowing it to work as a wireless controller for the IPG 10 or ETS 40, as described in U.S. Patent Application Publication 2015/0231402. RC 45 includes a user interface, including means for entering commands (e.g., buttons or icons) and a display 46. The RC 45's user interface enables a patient to adjust stimulation parameters, although it may have limited functionality when compared to the more-powerful CP 50.

In some examples, the RC 45 can have one or more antennas capable of communicating with the IPG 10 and ETS 40. For example, the RC 45 can have a near-field magnetic-induction coil antenna 47a capable of wirelessly communicating with the coil antenna 26a or 42a in the IPG 10 or ETS 40. The RC 45 can also have a far-field RF antenna 47b capable of wirelessly communicating with the RF antenna 26b or 42b in the IPG 10 or ETS 40.

In some examples, the RC 45 can have control circuitry 48 such as a microprocessor, microcomputer, an FPGA, other digital logic structures, an Applicant Specific Integrated Circuit (ASIC), etc., which is capable of executing instructions an electronic device. Control circuitry 48 can for example receive patient adjustments to stimulation parameters, and create a stimulation program to be wirelessly transmitted to the IPG 10 or ETS 40.

The CP 50 can be as described further in U.S. Patent Application Publication 2015/0360038, and is only briefly explained here. The CP 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the CP 50 that are usually specific to its operation as a therapy controller, such as a communication “wand” 54, and a joystick 58, which are coupleable to suitable ports on the computing device 51, such as USB ports 59 for example.

The antenna used in the CP 50 to communicate with the IPG 10 or ETS 40 can depend on the type of antennas included in those devices. If the patient's IPG 10 or ETS 40 includes a coil antenna 26a or 42a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient's IPG 10 or ETS 40.

In an example where the IPG 10 or ETS 40 includes an RF antenna 26b or 42b, the wand 54, the computing device 51, or both, can likewise include an RF antenna 56b to establish communication with the IPG 10 or ETS 40 at larger distances. (Wand 54 may not be necessary in this circumstance). The CP 50 can also establish communication with other devices and networks, such as the Internet, either wirelessly or via a wired link provided at an Ethernet or network port.

To program stimulation programs or parameters for the IPG 10 or ETS 40, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device's non-volatile memory 68. One skilled in the art will additionally recognize that execution of the clinician programmer software 66 in the computing device 51 can be facilitated by control circuitry 70 such as a microprocessor, microcomputer, an FPGA, other digital logic structures, etc., which is capable of executing programs in a computing device. The control circuitry 70 can execute the clinician programmer software 66 to generate a therapy plan and rendering the GUI 64. The therapy plan (also referred to as a stimulation program) may include stimulation parameters chosen through the GUI 64 (e.g., electrode configurations and stimulation dosing parameters). The control circuitry 70 can enable communications via antennas 56a or 56b to communicate the therapy plan (e.g., stimulation parameters) to the patient's IPG 10. The IPG 10 may deliver electrostimulation in accordance with the therapy plan.

In an example, the therapy plan includes a sub-perception SCS plan comprising stimulation parameters with respective values that can be set by the user via the GUI 64. In some examples, the sub-perception SCS can include a sub-perception therapy program that utilizes a particular stimulation frequency, such as at a level below 100 Hz (or in some instances lower than 10 Hz) and a biphasic-symmetric pulse waveform comprising an active charge phase followed by an active recharge phase. Stimulation pulses in the sub-perception therapy can be defined by stimulation parameters such as stimulation amplitudes, pulse width, frequency, etc. In some examples, sub-perception therapy can be delivered in discreet chunks, or boluses of stimulation pulses. Each bolus comprises stimulation pulses delivered during a first duration, followed by a second stimulation-free duration before a next bolus of stimulation pulses are delivered. A user may use the GUI 64 to program therapy settings, such as electrode selection and configuration, stimulation parameter values including, for example, amplitudes, pulse width, frequency, pulse waveform, active or passive recharge mode for the sub-perception therapy, ON time (the first duration), OFF time (the second duration), and therapy duration, among others. In some examples, the user may use the GUI 64 to define criteria for, and initiate a process of, identifying the patient's usage pattern of stimulation, such as frequency, activation time, duration, and manner of using certain stimulation programs.

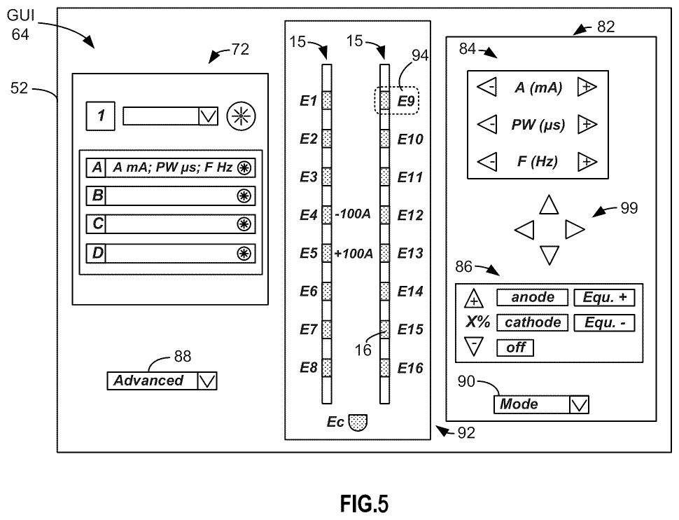

FIG. 5 illustrates, by way of example and not limitation, a portion of a GUI (such as one in a clinician programmer) for setting or adjusting stimulation parameters, such as the GUI 64 as shown in FIG. 4. One skilled in the art will understand that the particulars of the GUI 64 will depend on where clinician programmer software 66 is in its execution, which will depend on the GUI selections the clinician has made. FIG. 5 shows the GUI 64 at a point allowing for the setting of stimulation parameters for the patient and for their storage as a stimulation program. To the left a program interface 72 is shown, which as explained further in the '038 Publication allows for naming, loading and saving of stimulation programs for the patient. Shown to the right is a stimulation parameters interface 82, in which specific stimulation parameters (A, D, F, E, P) can be defined for a stimulation program. Values for stimulation parameters relating to the shape of the waveform (A; in this example, current), pulse width (PW), and frequency (F) are shown in a waveform parameter interface 84, including buttons the clinician can use to increase or decrease these values.

Stimulation parameters relating to the electrodes 16 (the electrodes E activated and their polarities P), are made adjustable in an electrode parameter interface 86. Electrode stimulation parameters are also visible and can be manipulated in a leads interface 92 that displays the leads 15 (or 15′) in generally their proper position with respect to each other, for example, on the left and right sides of the spinal column. A cursor 94 (or other selection means such as a mouse pointer) can be used to select a particular electrode in the leads interface 92. Buttons in the electrode parameter interface 86 allow the selected electrode (including the case electrode, Ec) to be designated as an anode, a cathode, or off. The electrode parameter interface 86 further allows the relative strength of anodic or cathodic current of the selected electrode to be specified in terms of a percentage, X. This is particularly useful if more than one electrode is to act as an anode or cathode at a given time, as explained in the '038 Publication. In accordance with the example waveforms shown in FIG. 2, as shown in the leads interface 92, electrode E5 has been selected as the only anode to source current, and this electrode receives X=100% of the specified anodic current, +A. Likewise, electrode E4 has been selected as the only cathode to sink current, and this electrode receives X=100% of that cathodic current, −A.

The GUI 64 as shown specifies only a pulse width PW of the first pulse phase 30a. The clinician programmer software 66 that runs and receives input from the GUI 64 will nonetheless ensure that the IPG 10 and ETS 40 are programmed to render the stimulation program as biphasic pulses if biphasic pulses are to be used. For example, the clinician programming software 66 can automatically determine durations and amplitudes for both of the pulse phases 30a and 30b (e.g., each having a duration of PW, and with opposite polarities +A and −A). An advanced menu 88 can also be used (among other things) to define the relative durations and amplitudes of the pulse phases 30a and 30b, and to allow for other more advance modifications, such as setting of a duty cycle (on/off time) for the stimulation pulses, and a ramp-up time over which stimulation reaches its programmed amplitude (A), etc. A mode menu 90 allows the clinician to choose different modes for determining stimulation parameters. For example, as described in the '038 Publication, mode menu 90 can be used to enable electronic trolling, which comprises an automated programming mode that performs current steering along the electrode array by moving the cathode in a bipolar fashion. While GUI 64 is shown as operating in the CP 50, the user interface of the RC 45 may provide similar functionality.

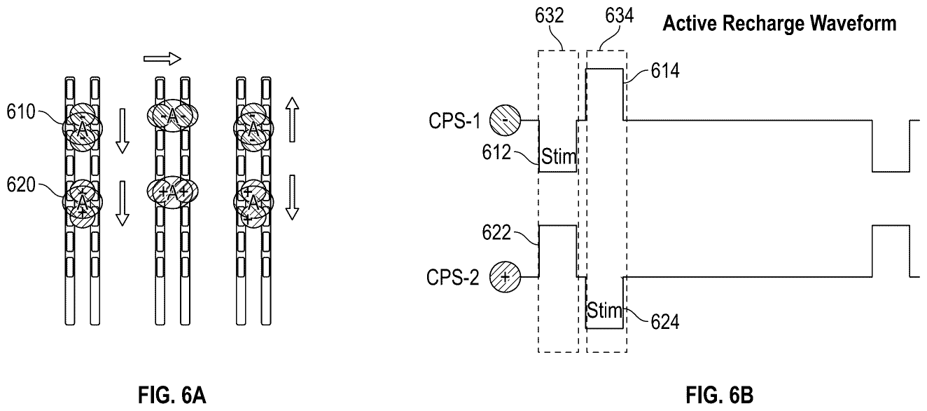

FIGS. 6A-6B illustrate, by way of example and not limitation, schematics of electrode configurations and stimulation waveforms that may be used in a sub-perception neuromodulation therapy. The sub-perception therapy can be programmed using a model-based steering algorithm that enables multiple central points of stimulation (CPS) to be moved rostrocaudally and mediolaterally simultaneously at a programmable step set by a user, as illustrated in FIG. 6A. In the illustrated example, by using symmetric biphasic waveforms, two separate CPSs can be implemented in the stimulation paradigm, including CPS-1 610 representing a virtual cathode, and CPS-2 620 representing a virtual anode. CPS-1 610 and CPS-2 620 can respectively sink or source various percentages of total current across multiple electrodes on the lead (also referred to as “current fractionalization”). For example, current applied to the virtual cathode CPS-1 610 can be fractionalized over a plurality of physical cathodes. Similarly, current applied to the virtual anode CPS-2 620 can be fractionalized over a plurality of physician anodes. The bipolar distance between the CPS-1 610 and CPS-2 620 can be programmed to be within a specified range, such as 10-14 mm. In an example, the bipolar distance is set to approximately 12 mm. The bipolar distance controls the spread of paresthesia during neural target search.

FIG. 6B illustrates biphasic symmetric waveforms of stimulation current for the virtual cathode CPS-1 610 and the virtual anode CPS-2 620. The biphasic symmetric waveform comprises a first charge phase 632, followed by a second active recharge (or charge recovery) phase 634. Current amplitude in each phase remains constant, thus a rectangular waveform. For each of the virtual cathode or the virtual anode, current amplitude of the charge phase 632 has the same magnitude but different sign (representing direction of current flow) than the current amplitude of the recharge phase 634. During the first rectangular phase 632, a negative current 612 (i.e., cathodic current) is injected through negatively configured contacts (physical cathodes corresponding to the virtual cathode CPS-1 610), and positive current 622 (i.e., anodic current) is injected through positively configured return contacts (physical anodes corresponding to the virtual anode CPS-2 620). During the second rectangular phase 634, the polarities of the virtual cathode CPS-1 and the virtual anode CPS-2 are reversed to achieve active charge balance: positive current 614 (i.e., anodic current) is applied to the assigned physical cathodes corresponding to virtual cathode CPS-1 610, and negative current 624 (i.e., cathodic current) is applied to the assigned physical anodes corresponding to virtual anode CPS-2 620.

Stimulation dosing parameters, such as amplitude, frequency (or stimulation rate), pulse width (PW), or waveform pattern of the stimulation waveform are programmable and can be set or adjusted by a user on a GUI. In an example, the frequency of the stimulation pulse (reciprocal of period) can be programmed to a value within a specific range, such as approximately 2-700 Hz. In an example, the stimulation frequency can be programmed to 90 Hz. The pulse width (PW) can be programmed within a range, such as approximately 210±50 micro-seconds (μs). To identify the electrode configuration and fine-tune the location of stimulation, a neural target search can be carried out using the CPS-1 and CPS-2 steered simultaneously in the rostro-caudal and medial-lateral dimensions at a programmable step (resolution) such as in approximately 300 us increments. The stimulation amplitude can then be lowered to a programmable fraction of the perception threshold. Such a programming for sub-perception therapy allows for a systematic optimization of the stimulating field that provides comprehensive overlap between the area of pain and paresthesia sensation.

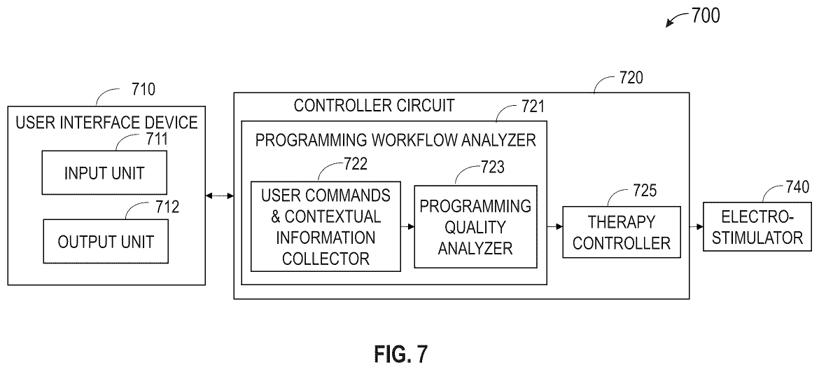

FIG. 7 illustrates, by way of example and not limitation, a neuromodulation system 700 configured to provide or adjust a neuromodulation therapy in accordance with parameters programmed through a computer-assisted individualized programming workflow. The system 700 can provide closed-loop neurostimulation to treat or alleviate various neurological or autonomic disorders or other physiological condition. In an example, the system 700 can provide closed-loop spinal cord stimulation (SCS) at a spinal neural target. The SCS can be a part of pain management regimen. In another example, the system 700 can provide closed-loop deep brain stimulation (DBS) at a brain target. The DBS can be used to treat various neurological disorders including, for example, dystonia, epilepsy, essential tremor, or Parkinson's disease. In some examples, the system 700 can titrate neurostimulation to treat or alleviate certain autonomic disorders.

The neuromodulation system 700, which is an embodiment of the neuromodulation system 400, may include one or more of a user interface device 710, a controller circuit 720, and an electrostimulator 740. Portions of the neuromodulation system 700 may be implemented in stimulation devices such as IPG 10 or the ETS 40, and/or controller devices such as the RC 45 or CP 50.

The user interface device 710 can allow a user (e.g., a clinician or a device expert) to program or modify a therapy setting, and to provide feedback on a neuromodulation therapy. In an example, the user interface device 710 can be a portable (e.g., handheld) device, such as the RC 45 or a smartphone (with executable software application) operable by the patient at his or her home without requiring extra clinic visits or consultation with a device expert. In another example, the user interface device 710 can be a programmer device, such as the CP 50, that allows a physician to review therapy settings and treatment history, consult with the patient to obtain feedback information (e.g., pain relief and side effects or symptoms), programming the electrostimulator 740, or provide other treatment options to the patient. The feedback can include, for example, therapeutic effectiveness and symptoms or side effects experienced by the patient during or after the neuromodulation therapy. Such feedback, in some examples, can include physiological signals sensed by physiological sensors in response to therapy delivered to the patient in accordance with a programmed candidate therapy program.

The user interface device 710 can include an input unit 711 and an output unit 712. The input unit 711 can include one or more user interface (UI) control elements operable by a user to program or modify a device setting. For example, the user may use the UI control elements to provide or adjust values for one or more programmable parameters including, for example, a signal acquisition parameter, a signal analysis parameter, or a neuromodulation therapy parameter, or to select from a plurality of pre-defined stimulation programs a desired simulation program for future use. Each stimulation program can include a number of stimulation parameters with respective pre-determined values.

In some examples, the input unit 711 can include UI control elements operable by the user to provide feedback on a neuromodulation therapy delivered to the patient. A therapy setting may include a set of stimulation parameters with respective programmable or preset values. Examples of the stimulation parameters can include an electrode configuration (e.g., stimulation lead and electrode location, selection of active electrodes, designation of anode and cathode, and stimulation current or energy fractionalization across the electrodes), stimulation dose parameters (e.g., pulse width, frequency, pulse amplitude), stimulation pulse waveform, or an ON-OFF cycling of stimulation bursts, among others. The feedback provided by the user via the input unit 711 can include pain data or feedback on pain relief by the existing SCS therapy. The pain data or the feedback on pain relief may include identification of pain sites, distribution of the pain, intensity of pain at various pain sites, or temporal pattern such as persistence of the pain at various pain sites, a pain drawing with pain markings identifying the locations, intensities, patterns of pain, among other information. In some examples, the feedback may include side effects or symptoms arise or persist associated with the SCS, or severity of the symptom or a side effect. The severity can take a numerical value (e.g., on a 1 to 5 scale) or a categorical value (e.g., “Mild”, “Moderate”, or “Severe”). The feedback may be provided in different formats, such as texts, graphs, or verbal descriptions, among others. In some examples, the user may provide, via the input unit 711, information about patient health or medical information, such as change in medication, physical activities, medical procedures received, among other information. As will be discussed further below, the therapy controller 725 may use patient feedback on the neuromodulation therapy, along with other contextual information and user commands during a programming session, to adjust a therapy setting or schedule.

The output unit 712 can include a display to present textually or graphically an interface for programming or modifying the device setting, and the information provided by the user via the input unit 711. In some examples, under the control of the controller circuit 720, the output unit 712 may display a recommendation to the user to program a neuromodulation therapy in accordance with an individualized programming workflow. The user may be prompted to select or deselect, or to change value(s) of, one or more programmable parameters on the user interface. In an example, the output unit 712 may present to the user an “optimal” therapy setting that includes recommended values for one or more device parameters. The user can accept, reject, or modify the therapy setting via the input unit 711. In some examples, the output unit 712 may display a progress of an ongoing neuromodulation therapy in real time.

The controller circuit 720, which is an example of the control circuitry 48 of the RC 45 or the control circuitry 70 of the CP 50, can be communicatively coupled to or integrated into the user interface device 710. The controller circuit 720 can generate an individualized programming workflow to guide the user to test and search for an optimal therapy setting (as defined by a set of stimulation parameters with respectively optimized values) that would achieve a desired therapeutic effect while avoiding or minimizing the side effects or symptoms, and to program the optimal therapy setting to the electrostimulator 740. The controller circuit 720 can include circuit sets comprising one or more other circuits or sub-circuits, such as a programming workflow analyzer 721 and a therapy controller 725. The programming workflow analyzer 721 may further include subcircuits including a user commands and contextual information collector 722 and a programming quality analyzer 723. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 720 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

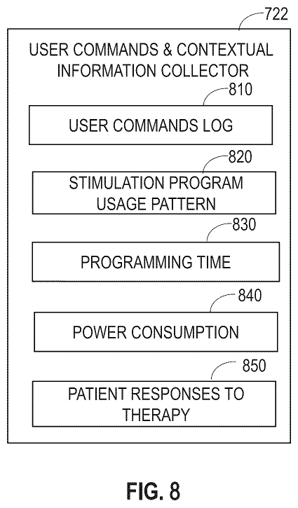

The user commands and contextual information collector 722 can collect information during a programming session, including a sequence of programming instructions for programming the electrostimulator 740, and contextual information in relation to how the programming instructions were made during a programming session. Referring to FIG. 8, by way of example and not limitation, examples of user commands and contextual information collected during the programming session (and used for generating an individualized programming workflow) may include one or more of a user commands log 810, a stimulation program usage pattern 820, programming time 830, power consumption 840, or patient responses to therapy 850. The user commands log 810 may include information about the programmable parameters that have been selected by the user during the programming session, such as UI control elements being activated (e.g., button press, screen touch, or mouse click) to select programmable parameters for signal acquisition and processing, feature extraction, or neuromodulation therapy configuration and delivery. The user commands log 810 may further include information about how such selections were made including, for example, a timing or timestamp for each selection, a duration of time spend on selecting or programming a parameter, a frequency of (i.e. how often) a parameter being selected over a specific time period or over a number of programming sessions, or an order of setting multiple programmable parameters in a programming session, such as button presses in an order of A-C-B-E-D where each button press selects one of programmable parameters “A” through “E”. The stimulation program usage pattern 820 represents a pattern of past usage of a particular stimulation program. The usage pattern can be provided by the user via the user interface device 710, or from a stored record of past stimulation usage. In an example, the information about the patient's past usage includes timing or duration for activating a particular stimulation program during a specific past time period, which can be specified by the user via the user interface device 710. The usage pattern 820 can be analyzed periodically or in an on-demand fashion. The programming time 830 represents time elapsed during an entirety or a portion of the programming session, such as time spent when the programmer device is in active communication with the electrostimulator 740 during the programming session. The power consumption 840 represents an estimate of power consumed during an entirety or a specific portion of the programming session, such as the power consumed when the programmer device is in active communication with the electrostimulator 740 during the programming session.

The user commands and contextual information collector 722 may additionally collect information about patient response to therapy 850 indicative of a therapeutic effect or a side effect of the neuromodulation therapy. The patient response to therapy 850 may be sensed using one or more sensors. Such sensors may be included in, or external to, the IPG 10 or the ETS 40, to sense a physiological signal. In an example, the user commands and contextual information collector 722 can be coupled to one or more subcutaneous electrodes on one or more implantable leads, such as the electrodes 16 on one or more electrode leads 15 as illustrated in FIG. 1, to sense a physiological signal. The sensed physiological signal represents evoked response to electrostimulation of a neural target delivered from the electrostimulator 740 (e.g., included in the IPG 10) via an electrode lead such as one or more leads 15. The electrostimulation pulses may be generated and delivered to the neural target in accordance with one or more stimulation parameters. Examples of the stimulation parameters can include stimulation waveform dosing parameters such as amplitude (e.g., current amplitude), pulse width, pulse rate or frequency, pulse pattern, pulse waveform, among others. The stimulation parameters may also include electrode configurations that define polarities of the electrodes used for delivering stimulation and fractionalization of current or electrical energy among the electrodes. In some examples, a stimulation pulse train may be delivered in accordance with a pre-defined stimulation program, such as a sub-perception therapy as described above with reference to FIGS. 6A-6B. The evoked response can be sensed from one or more of a dorsal column, a dorsal root, or a peripheral nerve. In some examples, the evoked responses can be somatosensory evoked potential (SSEP) signal recorded by electrodes placed on patient scalp over the sensory area of the brain in response to stimulation of specific nerves in, for example, ankle, wrist, or other external body parts. In an example, a biopotential signal can be sensed by one or more subcutaneous electrodes. The biopotential signal can include an evoked potential or evoked compound action potential (ECAP). The evoked responses (corresponding to the stimulation pulses) can include a plurality of inter-pulse segments of a biopotential signal.

The sensed physiological signal may additionally or alternatively include cardiac, pulmonary, neural, biochemical, or other physiological signals. Some of these signals may reveal characteristic signal properties in response to an onset, intensity, severity, duration, or patterns of pain. Examples of sensor signals can include cardiac signals such as a heart rate signal, a pulse rate signal, a heart rate variability signal, electrocardiogramar intracardiac electrogram, cardiovascular pressure signal, or heart sounds signal, among others. The second signal may additionally or alternatively include a galvanic skin response (GUR) signal, an electrodermal activity (EDA) signal, a skin temperature signal, an electromyogram (EMG) signal, an electroencephalogram (EEG) signal, a magnetoencephelogram (MEG) signal, a hemodynamic signal such as a blood flow signal, a blood pressure signal, a blood perfusion signal, a photoplethysmography (PPG) signal, or a saliva production signal indicating the change of amount of saliva production, among others.

In some examples, the patient response to therapy 850 may include information about body motion (e.g., bradykinesia, tremor, or rigidity), which is particularly useful for evaluating neuromodulation therapy in patients with Parkinson's disease. The body motion may be sensed using a motion sensor operably positioned on a leg or other body parts of the patient to sense the body motion, a sleep sensor attached to the patient or on a bed to sense body movement during sleep, or an imaging sensor (e.g., a camera or video recorder) for capturing images of body motion. In some examples, the sensors may be configured to sense information related to cognitive ability such as in patients with Alzheimer's disease. Examples of such sensors may include an audio sensor or an imaging sensor to record patient's verbal response or facial expressions in response to the electrostimulation delivered in accordance with candidate therapy settings. In some examples, the audio and/or the imaging sensor may be automatically activated to record patient verbal response or facial expressions when the patient is making a phone call or a video call, during which the electrostimulation is delivered in accordance with the candidate therapy settings.

In some examples, the user commands and contextual information collector 722 can be communicatively coupled to an imaging system such as a video camera that takes video or images of the user and/or the patient during a programming session. The user commands and contextual information collector 722 can generate video or image analytics indicative of user programming commands and contextual information. One or more of the user commands log 810, the stimulation program usage pattern 820, the programming time 830, the power consumption 840, or the about patient response to therapy 850 can be determined from the video or image analytics, which can subsequently be used to assessing the user's programming quality and to determine an optimal individualized programming workflow.

Referring back to FIG. 7, the programming quality analyzer 723 can determine a programming quality indicator using the collected user commands and contextual information from the collector 722. The programming quality indicator indicates, for a programming workflow, therapy optimality that would result from such programming and programming efficiency to achieve said therapy optimality. The programming quality indicator can take a numerical value or a categorical value. In an example, the programming quality indicator can be determined based on whether one or more of the user commands and contextual information satisfy respective conditions. For example, a high programming quality may be determined if one or more of the following conditions are satisfied: the user commands log 810 indicates the selection of parameters (e.g., button presses) are made in an order consistent with a predetermined optimal order; the stimulation program usage pattern 820 indicates a desired pattern of using a particular stimulation program (e.g., the number of activations within a monitoring period exceeds a threshold); the programming time 830 in a programming session falls below a time threshold; the power consumption 840 in a programming session falls below a power threshold; or the patient response to therapy 850 satisfies a therapeutic benefits and/or side effects requirement. In some examples, the programming quality indicator can take the form of a programming quality score. The programming quality score is a quantitative measure of the user's programming workflow, and can be determined using a weighted combination of some or all of the user commands log 810, the stimulation program usage pattern 820, the programming time 830, the power consumption 840, or the patient response to therapy 850.

In some examples, the programming quality analyzer 723 can perform a best-case and/or worst-case workflow analysis to assess risks associated with a particular user programming action, and determine an improvement in programming efficiency that can be made based on the risk analysis. In an example, fuzzy reasoning techniques may be used to determine a human factors index that may further be used to determine association rules between a programming workflow (e.g., a sequence of programming instructions such as button presses on the user interface) and a corresponding programming quality including therapy efficacy. In some examples, error producing conditions can be identified from the user's programming workflow, and quantified to assess human reliability regardless of actual workflow taken by the user. Worst-case workflows can be determined based on different combinations of programming instructions on the user interface.

In some examples, the programming quality analyzer 723 can perform workflow analysis and determine the programming quality indicator using artificial intelligence (AI) or machine learning (ML) methods. At least one ML model can be trained to establish association rules between a programming workflow (e.g., a sequence of programming instructions such as button presses) and a corresponding programming quality indicator. Supervised or unsupervised learning algorithms may be used to train the at least one ML model. Supervised learning uses prior knowledge (e.g., examples that correlate inputs to outputs or outcomes) to learn the relationships between the inputs and the outputs. The goal of supervised learning is to learn a function that, given some training data, best approximates the relationship between the training inputs and outputs so that the ML model can implement the same relationships when given inputs to generate the corresponding outputs. Some examples of commonly used supervised-ML algorithms are Logistic Regression (LR), Naive-Bayes, Random Forest (RF), neural networks (NN), deep neural networks (DNN), matrix factorization, and Support Vector Machines (SVM). Examples of DNN include a convolutional neural network (CNN), a recurrent neural network (RNN), a deep belief network (DBN), or a hybrid neural network comprising two or more neural network models of different types or different model configurations. Unsupervised learning is the training of an ML algorithm using information that is neither classified nor labeled, and allowing the algorithm to act on that information without guidance. Unsupervised learning is useful in exploratory analysis because it can automatically identify structure in data. Some common tasks for unsupervised learning include clustering, representation learning, and density estimation. Some examples of commonly used unsupervised learning algorithms are K-means clustering, principal component analysis, and autoencoders. In an example of training a ML model to predict an optimal filter setting (or other signal processing parameter) based on physiological signals acquired under different patient conditions, a training date set can be constructed using patient population data of physiological signals collected under different patient conditions and the filter settings that are demonstrated to provide optimal or desired performance distinguishing therapy effects under different patient conditions. Once trained, the ML model can predict the programming quality, including therapy outcome, for a given user programming workflow (e.g., a sequence of user programming instructions).

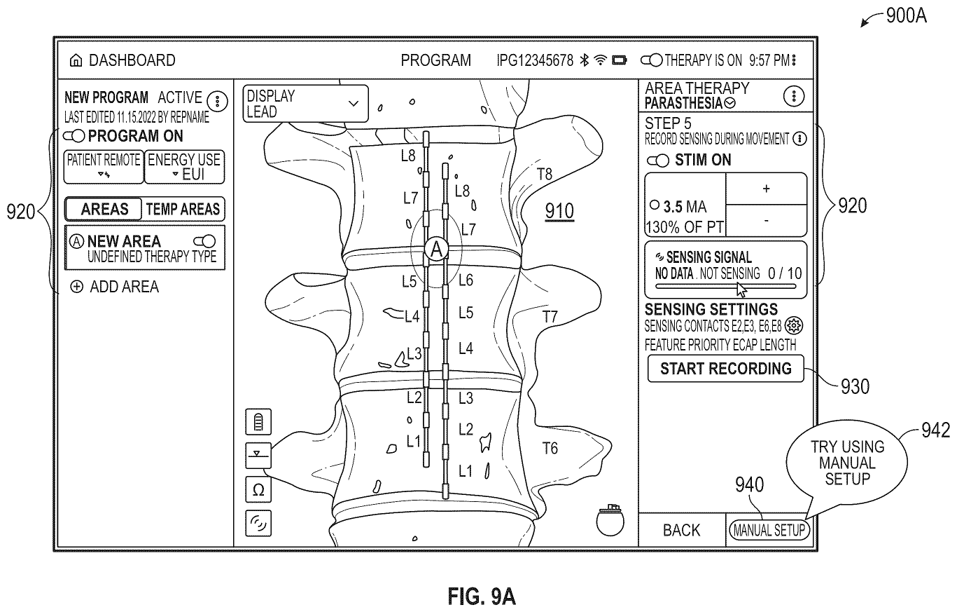

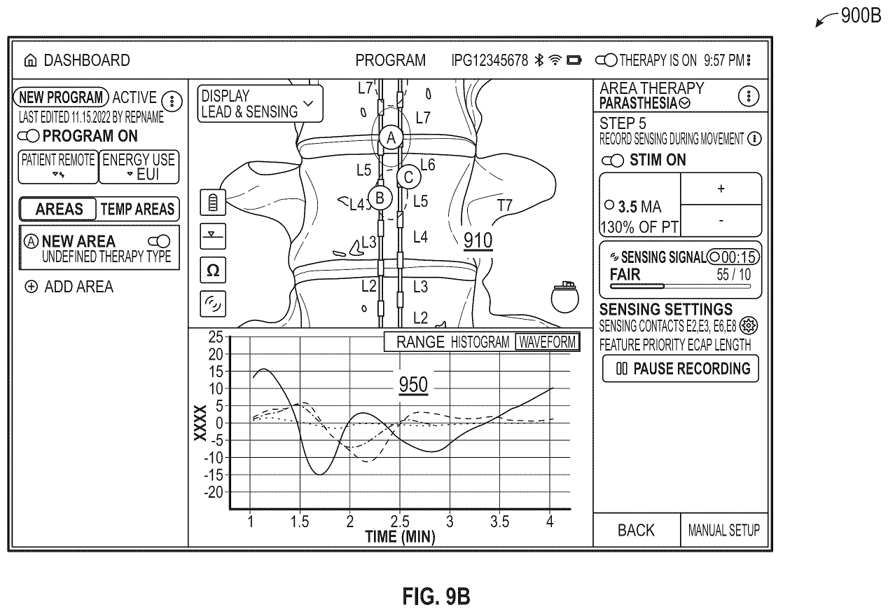

The therapy controller 725 can generate an individualized programming workflow based at least in part on the programming quality indicator. The individualized programming workflow may include various graphical user interface (GUI) designs on the user interface. In an example, the individualized programming workflow may include prompted or recommended selection (e.g., via buttons or other UI control element) of one or more programmable parameters that have not been or rarely been used by the user. Examples of the individualized programming workflow including recommended selection of programmable parameters are discussed below with respect to FIGS. 9A-9B.

In an example, the individualized programming workflow may include a shortcut parameter that enables simultaneous activation or selection of multiple parameters (e.g., one button press that effectively performs multiple selections simultaneously), thereby enabling quicker programming and optimizing programming performance. In another example, the individualized programming workflow may include bookmarking or highlighting “typical” selections or “frequently selected features” based on the user's past programming patterns.

The individualized programming workflow may include a prioritized order of selecting multiple programmable parameters (e.g., button presses in an order of A-B-C instead of B-A-C). In an example, the prioritized order can be determined based on the programmable parameters' respective dependencies on an active communication (e.g., Bluetooth, or Bluetooth Low Energy or BLE protocols) between the programmer device and the electrostimulator 740 during a programming session. The therapy controller 725 can identify a first group of programmable parameters that require active communication during the programming session, and a second group of programmable parameters that do not require active communication during the programming session. A batch-mode selection can be made such that the second group of programmable parameters that do not require active communication get to be selected prior to the first group of programmable parameters that require active communication. The grouping of the device parameters and the batch-mode selection of device parameters as described herein can improve the programming efficiency and reduce power consumption associated with active communication during a programming session.

In some examples, programmable features on the user interface can be arranged (e.g., grouped) based on user's past use patterns, or levels of familiarity with such features. The individualized programming workflow may include a group of “New Features” that the user typically does not use in past programming sessions, and a group of “Typical Features” that the user frequently use in past programming sessions. The “New features” can be grouped together and collectively shown in one area of the user interface, such as arranged under one tab. Similarly, the “Typical Features” can be grouped together and collectively shown in another area of the user interface, such as arranged under a different tab. In some examples, the individualized programming workflow may include a tutorial that goes through real examples of user workflows and how they progress through the various screens.

The therapy controller 725 can generate a control signal to the electrostimulator 740 to adjust the neuromodulation therapy based on the selected signal feature. The electrostimulator 740 can deliver a neuromodulation therapy in accordance with the adjusted therapy setting. Examples of the therapy setting may include, electrode selection and configuration, stimulation parameter values including, for example, amplitudes, pulse width, frequency, pulse waveform, active or passive recharge mode, ON time, OFF time, and therapy duration, among others. In an example, the therapy controller 725 can be implemented as a proportional integral (PI) controller, a proportional-integral-derivative (PID) controller, or other suitable controller that takes measurements of the selected signal feature (e.g., a “range” of epoch-averaged ECAP signal segments) as a feedback on the adjustment of therapy settings.

The electrostimulator 740 can be an implantable module, such as incorporated within the IPG 10. Alternatively, the electrostimulator 740 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 740 can deliver stimulation in accordance with the adjusted therapy setting.

FIGS. 9A-9B illustrate, by way of example and not limitation, at least a portion of a graphical user interface 900 that displays an individualized programming workflow to guide the user to test and program a device parameter to a neuromodulator that provides SCS therapy. The user interface 900 includes a display unit that displays programmable parameters and SCS lead placement 910 one a spinal cord target. A user may use UI controls 920 to program a therapy setting, such as by selecting a stimulation program or providing or adjusting a stimulation parameter value, and to command the electrostimulator to deliver a test stimulation therapy according to the programmed therapy setting. In this example, during the programming session the user keeps pressing “Start Recording” button 930 to collect neural signals from the spinal cord target, even though the sensed signal is continuously out of bounds. This behavior would not advance programming progress but continue drain the battery of the electrostimulator. The therapy controller 725, in detecting such user programming behavior, can generate a recommendation, such as a popup message or dialog box 942, to prompt the user to activate Manual Setup 940. FIG. 9B shows an updated user interface screen when the user presses the “Manual Setup” 940 button. The updated user interface shows programmable parameters that may be used for manually setting up sensing configurations and parameter values for sensing neural signals from the spinal cord, and a real-time or near real-time display of the neural signals 950 sensed from the spinal cord. In some examples, the updated user interface may additionally provide diagnostics of the user's programming workflow issues (e.g., in a message box), and provide a tutorial to guide the user to manually set up parameters for sensing neural signals from the spinal cord.

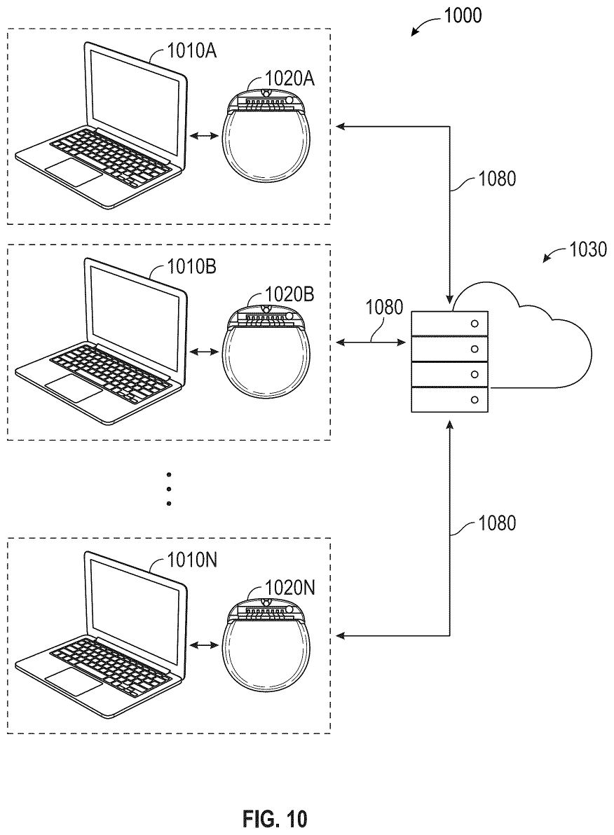

FIG. 10 illustrates an example of a cloud-based neuromodulation system 1000 configured to analyze multiple programming sessions and optimize workflows for programming respective electrostimulators across multiple patients. Each programming session involves a programmer device (one of 1010A, 1010B, . . . , 1010N, which can be CP 50 or RC 45) for programming an implantable electrostimulator (one of 1020A, 1020B, . . . , 1020N, which can be the IPG 10 or the electrostimulator 740). The programming sessions may be performed at different locations, such as different clinical facilities. Each of the programmer devices 1010A, 1010B, . . . , 1010N may include a user interface and a controller circuit, such as the user interface device 710 and the controller circuit 720 respectively as shown in FIG. 7. Each of the programmer devices 1010A, 1010B, . . . , 1010N can include a communication circuit to enable communication with a cloud computing system 1030 via a communication link 1080. Examples of the communication link 1080 may include Internet, LAN (Local Area Network), Wireless LAN (Wireless Local Area Network), WAN (Wide Area Network), PAN (Personal Area Network), or the like. The cloud computing system 1030 may include one or more servers that provide a suite of cloud-based services (e.g., software as a service, platform as a service, infrastructure as a service) allowing for secured, on-demand access by multiple clients including the programmer devices 1010A, 1010B, . . . , 1010N. The cloud services include, for example, collecting the information during the programming session and store the information in a cloud storage, determining the programming quality indicator, or generating an individualized programming workflow. In an example, the programmer devices 1010A, 1010B, . . . , 1010N can use one or more such cloud-based services to collect information during a programming session (such as one or more of those described in FIG. 8), analyze the workflow and generate a programming quality indicator, and generate a personalized workflow or make workflow recommendations. The cloud computing system 1030 allows for substantially concurrent analysis of multiple programming sessions at respective programmer devices and optimization of multiple programming workflows. Accordingly, faster individualized programming, enhanced reproducibility, and battery power savings can be achieved.

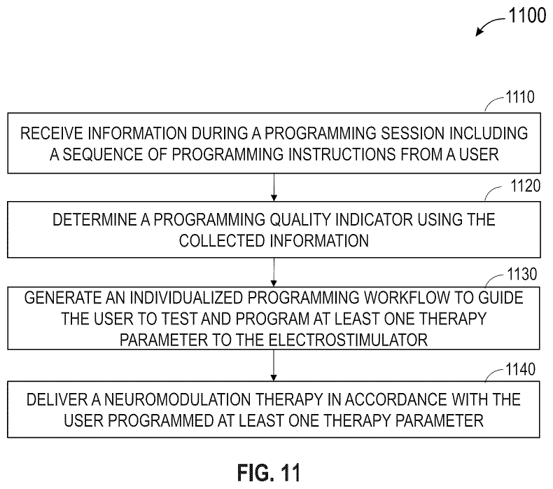

FIG. 11 is a flow chart illustrating, by way of example and not limitation, a method 1100 for programming an electrostimulator to provide electrostimulation to a patient. The method 1100 may be carried out using a medical system such as the neuromodulation system 400 or the neuromodulation system 700. For example, the method 1100 may be implemented in a programmer device such as RC 45 or CP 50 to test and program device parameters to an electrostimulator such as IPG 10 or electrostimulator 740. In an example, the method 1100 may be used to program and provide spinal cord stimulation (SCS) at a spinal neural target. In another example, the method 1100 may be used to provide closed-loop deep brain stimulation (DBS) at a brain target.

At 1110, during a programming session, user commands and contextual information including a sequence of programming instructions from a user can be received. The user can provide the programming instructions using one or more user interface (UI) control element on the user interface of the programmer device. The programming instructions may include selecting a programmable parameter, and providing or adjusting a value for the programmable parameter. Examples of the programmable parameter include a signal acquisition parameter, a signal analysis parameter, or a neuromodulation therapy parameter or stimulation program.

In addition to the sequence of programming instructions, contextual information associated with the user programming instructions may be received at 1110. Example of the contextual information include a timing or timestamp for each selection, a duration of time spend on selecting or programming a parameter, a frequency of (i.e. how often) a parameter being selected over a specific time period or over a number of programming sessions, or an order of setting multiple programmable parameters in a programming session. The contextual information may additionally or alternatively include a stimulation program usage pattern, time elapsed during an entirety or a portion of the programming session, such as time spent when the programmer device is in active communication with the electrostimulator during the programming session, or an estimate of power consumption during an entirety or a specific portion of the programming session, such as power consumed when the programmer device is in active communication with the electrostimulator during the programming session. In some examples, the user commands and contextual information may include information about patient responses to therapy indicative of a therapeutic effect or a side effect of the neuromodulation therapy. The patient responses may include an evoked neural response to electrostimulation of a neural target, physiological signals such as cardiac, pulmonary, neural, biochemical signals, information about body motion or information related to cognitive ability, among others.

At 1120, a programming quality indicator can be determined using the collected user commands and contextual information using various sources of the collected user commands and contextual information. The programming quality indicator indicates, for a programming workflow, therapy optimality that would result from such programming and programming efficiency to achieve said therapy optimality. The programming quality indicator can be determined based on whether one or more of the user commands and contextual information satisfy respective conditions. Alternatively, the programming quality can be represented by a numerical programming quality score computed using a weighted combination of various sources of the user commands and contextual information, such as those described with respect to FIG. 8. In some examples, a best-case and/or worst-case workflow analysis can be performed to assess risks associated with a particular user programming action, and an improvement in programming efficiency can be determined based on the risk analysis. In some examples, artificial intelligence (AI) or machine learning (ML) methods may be used to analyze the workflow and to determine the programming quality indicator.

At 1130, an individualized programming workflow can be generated based at least in part on the programming quality indicator. The individualized programming workflow can be used to guide the user to test and program at least one therapy parameter to the electrostimulator. In an example, the individualized programming workflow may include prompted or recommended selection of one or more programmable parameters that have not been or rarely been used by the user. In another example, the individualized programming workflow may include a shortcut parameter that enables simultaneous activation or selection of multiple parameters (e.g., one button press that effectively performs multiple selections simultaneously), thereby enabling quicker programming and optimizing programming performance. In yet another example, the individualized programming workflow may include bookmarking or highlighting “typical” selections or “frequently selected features” based on the user's past programming patterns. In some examples, the individualized programming workflow may additionally or alternatively include a prioritized order of selecting programmable parameters. In an example, the prioritized order can be determined based on the programmable parameters' respective dependencies on an active communication between the programmer device and the electrostimulator during a programming session. Programmable parameters that require active communication during the programming session and programmable parameters that do not require active communication during the programming session can be identified and grouped in separate groups. A batch-mode selection can be made such that the group of programmable parameters that do not require active communication get to be selected prior to the group of programmable parameters that require active communication.

At 1140, a neuromodulation therapy can be delivered to the patient using the electrostimulator in accordance with the user programmed at least one therapy parameter. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 740 can deliver stimulation in accordance with the adjusted therapy setting.

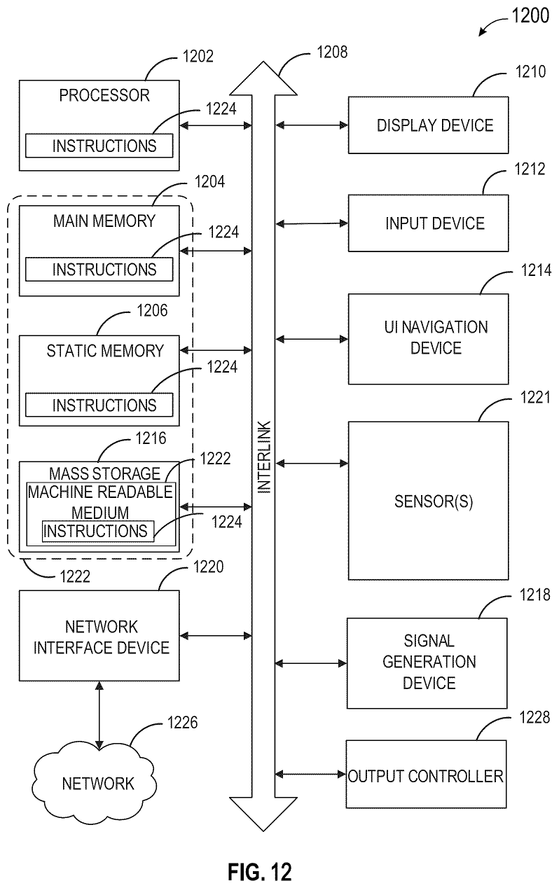

FIG. 12 illustrates generally a block diagram of an example machine 1200 upon which any one or more of the techniques (e.g., methodologies) discussed herein may perform. Portions of this description may apply to the computing framework of various portions of the neuromodulation device or the external programmer device.

In alternative examples, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1200 may include a hardware processor 1202 (e.g., a central processing unit (CPU), a graphics processing unit (GPU), a hardware processor core, algorithm specific ASIC, or any combination thereof), a main memory 1204 and a static memory 1206, some or all of which may communicate with each other via an interlink (e.g., bus) 1208. The machine 1200 may further include a display unit 1210 (e.g., a raster display, vector display, holographic display, etc.), an alphanumeric input device 1212 (e.g., a keyboard), and a user interface (UI) navigation device 1214 (e.g., a mouse). In an example, the display unit 1210, input device 1212 and UI navigation device 1214 may be a touch screen display. The machine 1200 may additionally include a storage device (e.g., drive unit) 1216, a signal generation device 1218 (e.g., a speaker), a network interface device 1220, and one or more sensors 1221, such as a global positioning system (GPS) sensor, compass, accelerometer, or other sensors. The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1216 may include a machine-readable medium 1222 on which is stored one or more sets of data structures or instructions 1224 (e.g., software) embodying or utilized by any one or more of the techniques or functions described herein. The instructions 1224 may also reside, completely or at least partially, within the main memory 1204, within static memory 1206, or within the hardware processor 1202 during execution thereof by the machine 1200. In an example, one or any combination of the hardware processor 1202, the main memory 1204, the static memory 1206, or the storage device 1216 may constitute machine readable media.

While the machine-readable medium 1222 is illustrated as a single medium, the term “machine readable medium” may include a single medium or multiple media (e.g., a centralized or distributed database, and/or associated caches and servers) configured to store the one or more instructions 1224.

The term “machine readable medium” may include any medium that is capable of storing, encoding, or carrying instructions for execution by the machine 1200 and that cause the machine 1200 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1224 may further be transmitted or received over a communication network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1220 may include one or more physical jacks (e.g., Ethernet, coaxial, or phone jacks) or one or more antennas to connect to the communication network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1200, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for providing electrostimulation to a patient, comprising:
an electrostimulator configured to provide a neuromodulation therapy to a patient; and
a programmer device operable by a user to program the electrostimulator, the programmer device including:
a user interface; and
a controller circuit configured to:
collect user commands and contextual information during a programming session including a sequence of programming instructions from the user via the user interface;
determine a programming quality indicator using the collected user commands and contextual information; and
based at least in part on the programming quality indicator, generate an individualized programming workflow to guide the user to test and program a therapy setting to the electrostimulator;
wherein the electrostimulator is configured to provide the neuromodulation therapy in accordance with the user programmed therapy setting.

2. The system of claim 1,
wherein the sequence of programming instructions from the user during the programming session includes one or more of a timing of a programmable parameter being selected, a frequency of a programmable parameter being selected over a specific time period, or an order of selecting multiple programmable parameters,
wherein the individualized programming workflow includes selection or deselection of a programmable parameter, or an order of selecting multiple programmable parameters.

3. The system of claim 1, wherein the controller circuit is configured to receive image or video data from an imaging system during the programming session, and to extract information about the sequence of programming instructions from the received image or video data.

4. The system of claim 1, wherein the user commands and contextual information collected during the programming session further includes time elapsed during an entirety or a portion of the programming session, including time spent when the programmer device is in active communication with the electrostimulator during the programming session.

5. The system of claim 1, wherein the user commands and contextual information collected during the programming session further includes an estimate of power consumption during an entirety or a specific portion of the programming session, including an estimate of power consumption when the programmer device is in active communication with the electrostimulator during the programming session.

6. The system of claim 5, wherein the individualized programming workflow includes an ordered selection of multiple programmable parameters based on their respective dependencies on an active communication during the programming session, such that a first group of programmable parameters that do not require active communication during the programming session are selected prior to a second group of programmable parameters that require active communication during the programming session.

7. The system of claim 1, wherein the user commands and contextual information collected during the programming session further includes patient responses indicative of a therapeutic effect or a side effect of the neuromodulation therapy.

8. The system of claim 1, wherein the controller circuit is configured to determine the programming quality indicator based on a comparison of the sequence of programming instructions to a reference sequence of programming instructions.

9. The system of claim 1, wherein the programming quality indicator includes a programming quality score computed using a weighted combination of various sources of the user commands and contextual information collected during the programming session.

10. The system of claim 1, wherein the controller circuit is configured to determine the programming quality indicator by applying a trained machine learning model to the user commands and contextual information collected during the programming session.

11. The system of claim 1, wherein the controller circuit is configured to present on the user interface the individualized programming workflow, including to prompt the user to select or deselect one or more programmable parameters, or to select multiple programmable parameters in a specific order.

12. The system of claim 11, wherein the programmable parameters include at least one of a signal acquisition parameter, a signal analysis parameter, or a neuromodulation therapy parameter.

13. The system of claim 1, wherein the programmer device includes a communication circuit to communicate with a cloud computing system, and to use one or more cloud services provided by the cloud computing system to perform one or more operations including collecting the user commands and contextual information during the programming session, determining the programming quality indicator, or generating the individualized programming workflow.

14. A method of programming an electrostimulator using a programmer device to provide electrostimulation to a patient, the method comprising:
receiving user commands and contextual information during a programming session, including a sequence of programming instructions from a user via a user interface;
determining a programming quality indicator using the user commands and contextual information;
based at least in part on the programming quality indicator, generating an individualized programming workflow to guide the user to test and program a therapy setting to the electrostimulator; and
delivering a neuromodulation therapy to the patient via the electrostimulator in accordance with the user programmed therapy setting.

15. The method of claim 14,
wherein the sequence of programming instructions from the user during the programming session includes one or more of a timing of a programmable parameter being selected, a frequency of a programmable parameter being selected over a specific time period, or an order of selecting multiple programmable parameters,
wherein the individualized programming workflow includes selection or deselection of a programmable parameter, or a specific order of selecting multiple programmable parameters.

16. The method of claim 14, wherein the user commands and contextual information during the programming session further includes at least one of:
time elapsed during an entirety or a portion of the programming session, including time spent when the programmer device is in active communication with the electrostimulator during the programming session; or
an estimate of power consumption during an entirety or a specific portion of the programming session, including an estimate of power consumption when the programmer device is in active communication with the electrostimulator during the programming session.

17. The method of claim 16, wherein the individualized programming workflow includes an ordered selection of multiple programmable parameters based on their respective dependencies on an active communication during the programming session, such that a first group of programmable parameters that do not require active communication during the programming session are selected prior to a second group of programmable parameters that require active communication during the programming session.

18. The method of claim 14, wherein the programming quality indicator includes a programming quality score computed using a weighted combination of various sources of the received user commands and contextual information during the programming session.

19. The method of claim 14, comprising presenting on the user interface the individualized programming workflow, including prompting the user to select or deselect a programmable parameter, or selecting multiple programmable parameters in a specific order,
wherein the programmable parameter includes at least one of a signal acquisition parameter, a signal analysis parameter, or a neuromodulation therapy parameter.

20. The method of claim 14, comprising:
establishing a communication between the programmer device and a cloud-computing system and receiving cloud-based services from the cloud-computing system; and
using the received cloud-based services to perform one or more operations including collecting the user commands and contextual information during the programming session, determining the programming quality indicator, or generating the individualized programming workflow.
